(19) United States
(12) Patent Application Publication
(10) Pub. No.: US 2014/0125953 A1
(43) Pub. Date: May 8, 2014

(54) Visual field visual function mapping apparatus

(71) Applicant: Tomohiro Tsuta, Kobe (JP)

(72) Inventor: Tomohiro Tsuta, Kobe (JP)

(21) Appl. No.: 13/998,465

(22) Filed: Nov. 4, 2013

(30) Foreign Application Priority Data
Nov. 8, 2012 (JP) 2012-259098

(51) Int. Cl. A61B 3/00; A61B 3/024

(52) U.S. Cl. CPC A61B 3/0025; A61B 3/024
USPC 351/224

(57) ABSTRACT

A visual field visual function mapping apparatus, with respect to the visual field mapping image obtained from the visual field scanning apparatus, including: along each concentric circle trajectory visual field mapping rectangle width average calculation means; and visual field mapping rectangle width average radial direction transition graph generating means.

### BACKGROUND OF THE INVENTION

The present invention relates to a visual field visual function mapping apparatus. There is no device that is a visual field visual function mapping apparatus and further includes means of the present invention.

The aim of the present invention is to provide a visual field visual function mapping apparatus, with respect to the visual field mapping image obtained from the use of the visual field scanning apparatus, characterized by further including:

along each concentric circle trajectory visual field mapping rectangle width average calculation means to calculate visual field mapping rectangle width average along each concentric circle trajectory by generating a cluster of concentric circles by incrementing the radius to generate a concentric circle in steps of a predetermined amount from the location on said visual field mapping image which corresponds to the fixation image display position at the time of said use of the visual field scanning apparatus;

and visual field mapping rectangle width average radial direction transition graph generating means to generate a graph which represents a transition from said location to the radial direction of visual field mapping rectangle width average along said each concentric circle trajectory based on the value of visual field mapping rectangle width average along said each concentric circle trajectory which is calculated by said along each concentric circle trajectory visual field mapping rectangle width average calculation means.

Another aim of the present invention is to provide a visual field visual function mapping apparatus, with respect to the visual field mapping image obtained from the use of the visual field scanning apparatus, characterized by further including:

for each quadrant along each concentric circle trajectory visual field mapping rectangle width average calculation means to calculate, for each quadrant of said visual filed mapping image whose origin is placed at the location on said visual field mapping image which corresponds to the fixation image display position at the time of said use of the visual field scanning apparatus, the visual field mapping rectangle width average along each concentric circle trajectory through generating a cluster of concentric circles by incrementing the radius to generate a concentric circle in steps of a predetermined amount from said location on said visual field mapping image which corresponds to the fixation image display position at the time of said use of the visual field scanning apparatus;

and for each quadrant visual field mapping rectangle width average radial direction transition graph generating means to generate, for each quadrant, a graph which represents a transition from said location to the radial direction of visual field mapping rectangle width average for said each quadrant along each concentric circle trajectory based on the value of visual field mapping rectangle width average for said each quadrant along each concentric circle trajectory which is calculated by said for each quadrant along each concentric circle trajectory visual field mapping rectangle width average calculation means.

### SUMMARY OF THE INVENTION

To achieve the above aim,

the invention of claim 1 is,

a visual field visual function mapping apparatus, with respect to the visual field mapping image obtained from the use of the visual field scanning apparatus, characterized by further including:

along each concentric circle trajectory visual field mapping rectangle width average calculation means to calculate visual field mapping rectangle width average along each concentric circle trajectory by generating a cluster of concentric circles by incrementing the radius to generate a concentric circle in steps of a predetermined amount from the location on said visual field mapping image which corresponds to the fixation image display position at the time of said use of the visual field scanning apparatus;

and visual field mapping rectangle width average radial direction transition graph generating means to generate a graph which represents a transition from said location to the radial direction of visual field mapping rectangle width average along said each concentric circle trajectory based on the value of visual field mapping rectangle width average along said each concentric circle trajectory which is calculated by said along each concentric circle trajectory visual field mapping rectangle width average calculation means.

To achieve the above aim,

the invention of claim 2 is,

a visual field visual function mapping apparatus, with respect to the visual field mapping image obtained from the use of the visual field scanning apparatus, characterized by further including:

for each quadrant along each concentric circle trajectory visual field mapping rectangle width average calculation means to calculate, for each quadrant of said visual filed mapping image whose origin is placed at the location on said visual field mapping image which corresponds to the fixation image display position at the time of said use of the visual field scanning apparatus, the visual field mapping rectangle width average along each concentric circle trajectory through generating a cluster of concentric circles by incrementing the radius to generate a concentric circle in steps of a predetermined amount from said location on said visual field mapping image which corresponds to the fixation image display position at the time of said use of the visual field scanning apparatus;

and for each quadrant visual field mapping rectangle width average radial direction transition graph generating means to generate, for each quadrant, a graph which represents a transition from said location to the radial direction of visual field mapping rectangle width average for said each quadrant along each concentric circle trajectory based on the value of visual field mapping rectangle width average for said each quadrant along each concentric circle trajectory which is calculated by said for each quadrant along each concentric circle trajectory visual field mapping rectangle width average calculation means.

### BRIEF DESCRIPTION OF THE DRAWINGS

FIG. 1 is a diagram showing a preferred embodiment of the present invention.

FIG. 2 is a diagram showing a preferred embodiment of the present invention.

FIG. 3 is a diagram showing a preferred embodiment of the present invention.

FIG. 4 is a diagram showing a preferred embodiment of the present invention.

FIG. 5 is a diagram showing a preferred embodiment of the present invention.

FIG. 6 is a diagram showing a preferred embodiment of the present invention.

FIG. 7 is a diagram showing a preferred embodiment of the present invention.

FIG. 8 is a diagram showing a preferred embodiment of the present invention.

FIG. 9 is a diagram showing a preferred embodiment of the present invention.

FIG. 10 is a diagram showing a preferred embodiment of the present invention.

FIG. 11 is a diagram showing a preferred embodiment of the present invention.

FIG. 12 is a diagram showing a preferred embodiment of the present invention.

FIG. 13 is a diagram showing a preferred embodiment of the present invention.

FIG. 14 is a diagram showing a preferred embodiment of the present invention.

FIG. 15 is a diagram showing a preferred embodiment of the present invention.

FIG. 16 is a diagram showing a preferred embodiment of the present invention.

FIG. 17 is a diagram showing a preferred embodiment of the present invention.

FIG. 18 is a diagram showing a preferred embodiment of the present invention.

FIG. 19 is a diagram showing a preferred embodiment of the present invention.

FIG. 20 is a diagram showing a preferred embodiment of the present invention.

FIG. 21 is a diagram showing a preferred embodiment of the present invention.

FIG. 22 is a diagram showing a preferred embodiment of the present invention.

FIG. 23 is a diagram showing a preferred embodiment of the present invention.

FIG. 24 is a diagram showing a preferred embodiment of the present invention.

FIG. 25 is a diagram showing a preferred embodiment of the present invention.

### DETAILED DESCRIPTION OF THE INVENTION

This is an invention of novel methodology and apparatus for visual field measurement. This is a patient-centered, novel and promising visual field measurement device discovered and invented by the inventor with visual defects.

Although very simple, this invention is practically very effective, possibly overwhelming existing visual field measurement devices in several functions.

This invention could easily realize very precise and detailed visual field examination unobtainable from the world's prevailing visual field measurement devices.

This invention could contribute to earlier detection of visual defects, as well as the eye care.

This invention can yield visual field mapping image that is utterly unique to this invention, strongly indicating (reflecting) the subject's retinal structure (possibly, retinal neural cell densities, photoreceptor cell densities, etc).

This invention would detect minute (subtle) visual defects associated with the minute nerve fiber layer abnormality, etc., which is not observable by other means. Its measurement could be instrumental in the early recognition of (glaucomatous) eye and optic nerve changes: This invention could be a very simple and handy early detection system of visual field alteration, glaucomatous development, etc.

For my proposal of this invention, the successive responses made by a subject via superficial subjectivity to the successively presented obvious (accustomed) stimuli can be extremely (excellently) objective in their nature with respect to visual function measurement (and also behavioral response time measurement).

(i.e., for my proposal of this invention, the successive responses made by a subject via superficial subjectivity to the successively presented reflexively understandable stimuli can be extremely (excellently) objective in their nature with respect to visual function measurement (and also to behavioral response time measurement).)

This invention could also be viewed as a method for very precise neurocognitive testing (on behavioral reaction time).

This invention, the new method of visual field measurement, is valuable not only from the medical point of view, but also from the visually impaired patients' point of view.

Incorporating (installing) this invention (program), an ordinary computer (or computerized display unit) can instantly turn into a novel, scientifically very instructive instrument with great performance for visual field measurement and mapping. The precise scan of the visual field can readily be realized on the display of an ordinary computer by this invention.

From the patients' point of view, this invention can enable patients to closely observe and correctly know their own (visual field) symptoms and to express (or print out) them to the outsides who frequently lack understanding to the patients' (visual) symptoms.

This invention could also be used as (glaucomatous) screening, (very precise and very reproducible) visual field self-check, etc.

And this invention could be used frequently in conducting visual field basic research with high precision and reproducibility (in laboratory).

And this invention can also be used binocularly by a subject to map a binocular visual field (function) with high precision and reproducibility.

This invention could contribute to an advancement of today's medical science and to the further accumulation of precise medical knowledge that seems to have been sparse in the science of visual field.

This invention could give rise to possibilities for discoveries and fresh educational experiences taking advantages of today's (personal) computer's strong points in explicitly visualizing the ordinarily unnoticed, regarding the visual function of the visual field.

There would exist global demand for this invention, since it could contribute to earlier detection of visual field defects, as well as to the eye-care.

### ON THE PROPOSED INVENTION

The discovery of novel methodology for visual field measurement.

The distinguished features of the proposed methodology are enumerated below.

The timing of scan line changing:

The visual field is successively scanned by a visual target along a scan line, through which a number of thresholds (relating to distance) are detected.

And a change of the scan line to a next scan line follows the completion of such successive scanning to the scan line.

(Realizing the detection of scotoma of subtlety, such as the early stage, glaucomatous scotoma and paracentral scotoma.)

The number of the thresholds detected along a scan line:

Not preliminarily fixed.

Flexible according to the visual sensitivity on a location under scanning of the visual field.

The number of thresholds being detected increases automatically to make close investigation at central visual field that is important to daily life, while at peripheral visual field, visual function declining region, and visual defects region, the number of thresholds being detected decreases automatically to make more rapid examination.

The interval between test points:

Flexible according to the visual sensitivity on a scanning location of the visual field.

The width of a visual field mapping rectangle is determined based on the distance traveled by a visual target from the time of the visual target's starting the kinetic scan to the time of (a response has been made by) the subject's perceiving such kinetic scan (i.e., the movement of the visual target) in the visual field.

The widths of the visual field mapping rectangles automatically decrease to make close investigation at the central visual field which is important to daily life, while at the peripheral visual field, visual function declining region, and visual defects region, the widths of the visual field mapping rectangles automatically increase to make more rapid examination.

The content of the threshold to be detected:

Based on (the distance traveled by a visual target) from the time of the visual target's starting the kinetic scan to the time of (a response has been made by) the subject's perceiving such kinetic scan (i.e., the movement of the visual target) in the visual field.

An end point of a scanning on a scan line:

An end point of a scanning on a scan line is a visual target's location at which the visual target's movement can be perceived (and responded to) by the subject in the visual field.

(At that location, the visual target stops the kinetic scan.)

A start point of a scanning on a scan line:

The end point of the last scan becomes the starting point of the next scan.

Realizing the exhaustive mapping of the visual field of visual sensitivity.

(Realizing the detection of scotoma of subtlety, such as the early stage, glaucomatous scotoma and paracentral scotoma.)

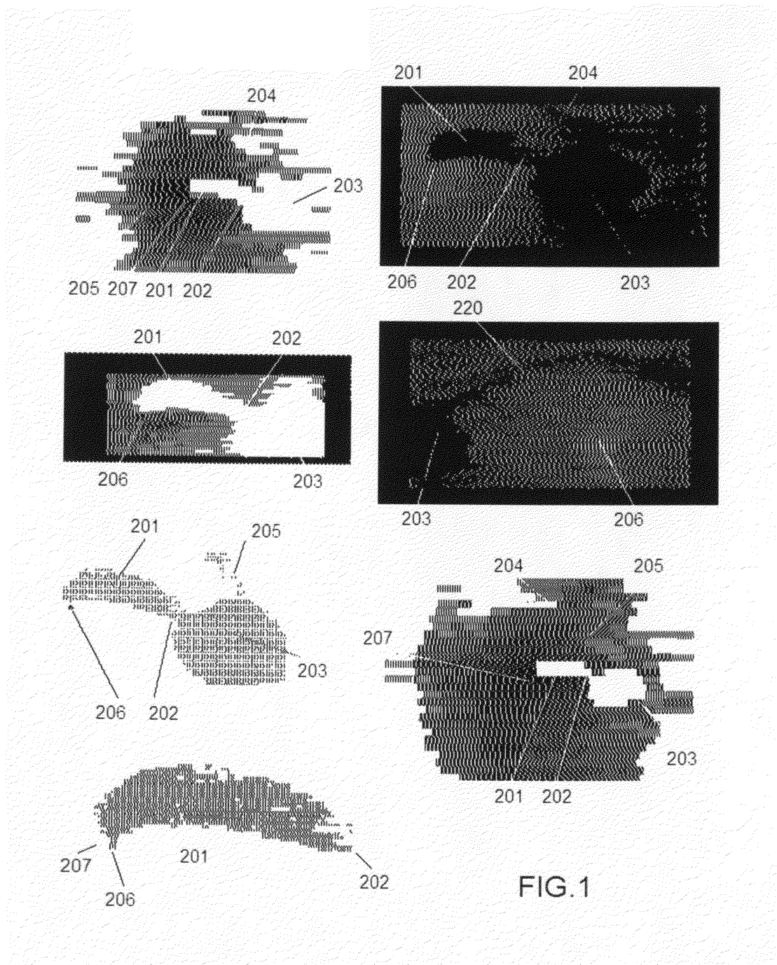

FIG. 1 is the real data (mapping images) of visual field measurement by this invention and related inventions.

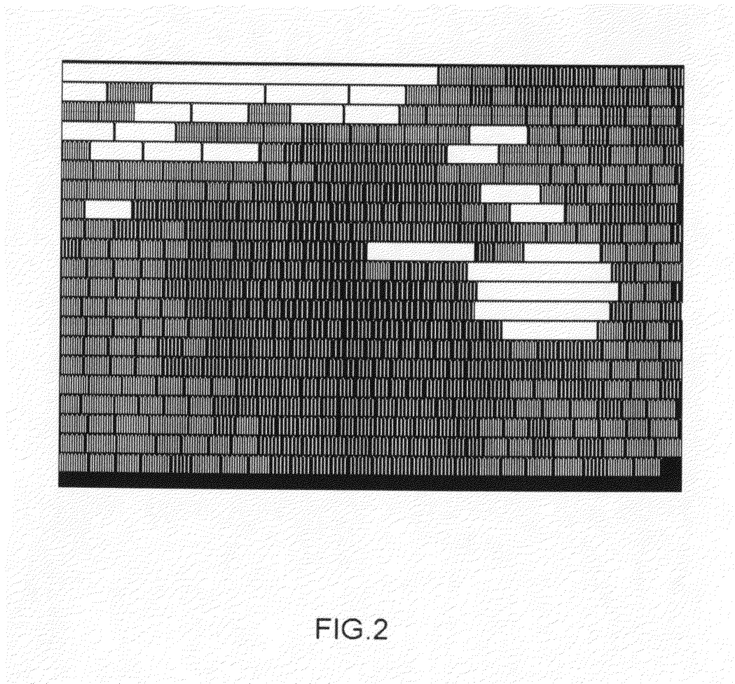

FIG. 2 is an example of right eye visual field mapping image obtained from the proposed invention. (viewing distance: about 31.6 cm)

The width of a visual field mapping rectangle is determined based on the distance traveled by a visual target from the time of the visual target's starting the (rightward) kinetic scan to the time of (the response of) the subject's perceiving such kinetic scan (i.e., the movement of the visual target) in the visual field.

The visual field mapping image obtained from the proposed invention is thought to strongly indicate (reflect) the subject's retinal structure (possibly, retinal neural cell densities, photoreceptor cell densities, etc).

FIG. 2 was obtained under the condition where

the computer display resolution was 1024*768 (in units of dot(pixel)),

the RGB color code for the visual field scanning screen background color was (for example) (0,0,0),

the size of the visual target was (for example) 5*5 (in units of dot(pixel))

(which corresponds to about 0.25 degrees*0.25 degrees in the case of fixation target viewing distance of about 31.6 cm),

the RGB color code of the visual target was (for example) (0,255,0)

(the visual target was a stimulus with suprathreshold intensity),

the velocity of the visual target movement was (for example) 10.515000 ms/dot, 4.755112 degs/s,

and

the observing distance of the fixation image (shown on the center of the display) was (for example) about 31.6 cm.

(The time required to obtain FIG. 2 by the visual field scan: 8.173183 minutes.)

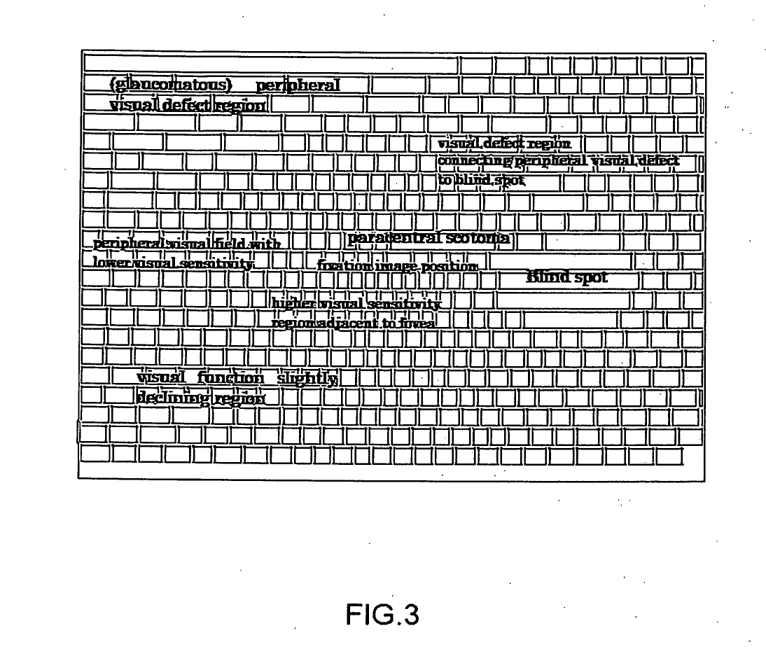

FIG. 3 is the explanation of the (right eye) visual field mapping image (of FIG. 2).

A (large) physiological blind spot is mapped in the temporal visual field.

(Glaucomatous) peripheral visual defect region is mapped in superior nasal visual field. A visual defect region connecting the peripheral visual defect to the blind spot is also mapped in the superior temporal visual field above the blind spot.

A paracentral scotoma is mapped at the central visual field.

The fixation image (display) position is shown at the center of the screen.

Higher visual sensitivity region adjacent to fovea is mapped by a cluster of rectangles with narrower widths and darker colors.

Peripheral visual field with lower visual sensitivity is mapped by a cluster of rectangles with wider widths and brighter colors.

A visual function slightly declining region is also mapped in the inferior nasal visual field.

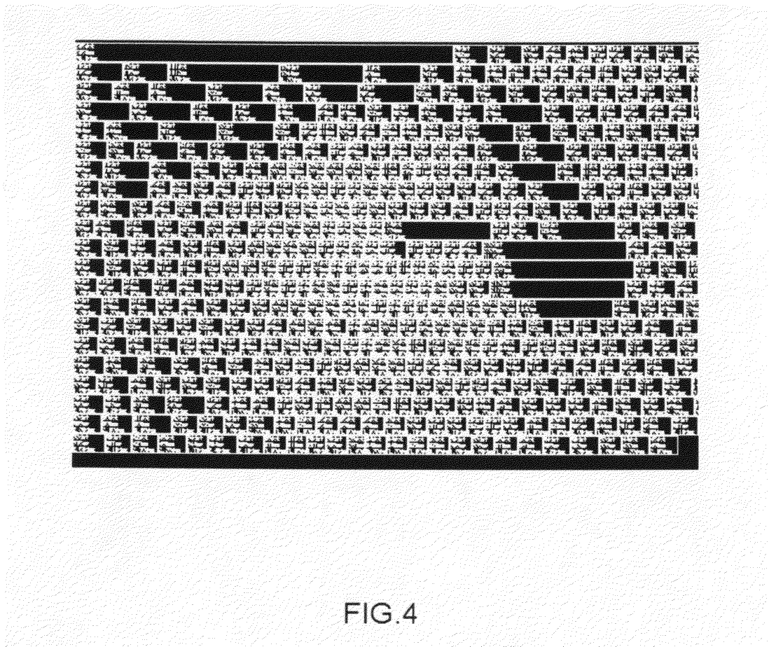

FIG. 4 is a numerical representation of the visual field mapping image (of FIG. 2

The width of each visual field mapping rectangle is denoted within each rectangle in dots.

Representation in degrees roughly calculated for viewing distance of around 31.6 cm and representation in milliseconds calculated as the time required for generating each visual field mapping rectangle are also denoted in each visual field mapping rectangle.

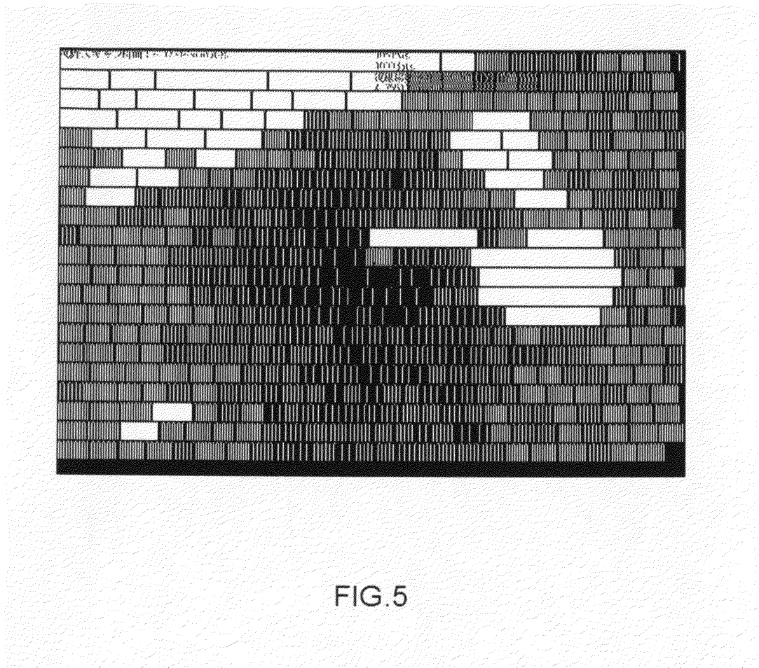

FIG. 5 is a simple enhancement (emphasis) of the visual field mapping image (of FIG. 2).

The visual sensitivity declining region is simply emphasized visually by subtracting around minimum (in terms of fovea proximity) rectangle width from each visual field mapping rectangle width before (linearly) converting each visual field mapping rectangle width into a brightness with which each visual field mapping rectangle is filled, and increasing (linear) scaling factor.

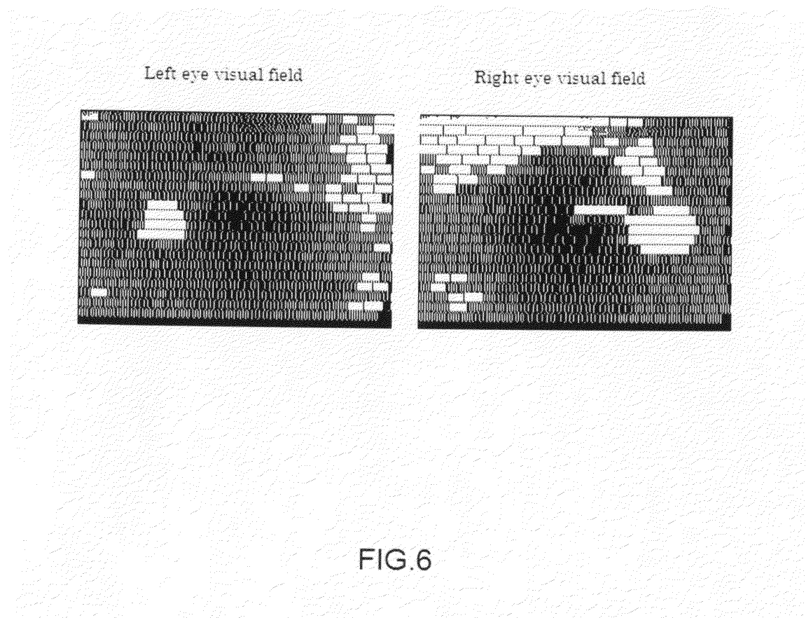

In FIG. 6, (corresponding) left eye visual field is juxtaposed to FIG. 5 (right eye visual field).

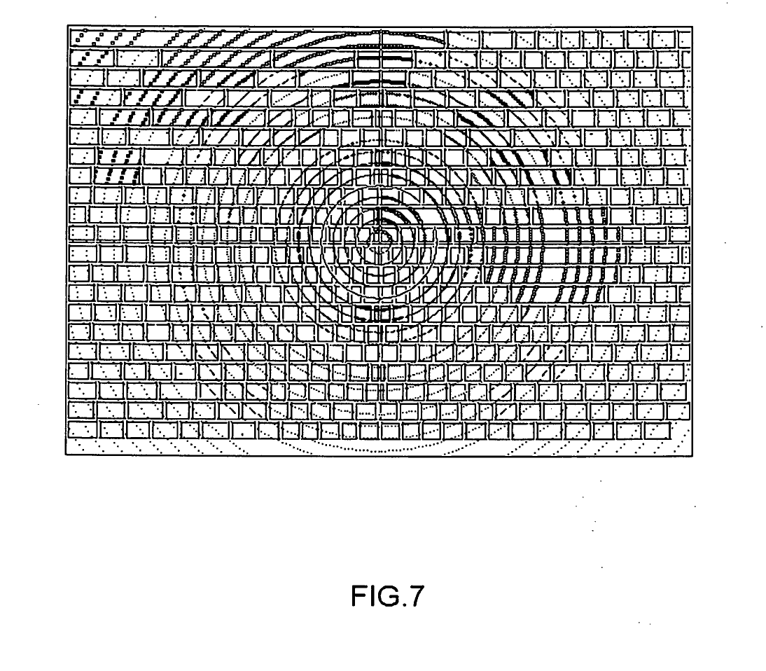

In FIG. 7, a series of concentric circles generated (by the CPU 501) by incrementing radius in steps of one degree (roughly calculated (by the CPU 501) via simple tangent calculation) from the fixation image display position (in this case, the center of the figure) are overlaid on the visual field mapping image (of FIG. 2).

(FIG. 2 is an example of the visual field mapping image obtained from the use of the visual field scanning apparatus.)

(CPU 501 generates a cluster of concentric circles (as shown in FIG. 7) by incrementing the radius to form a concentric circle in steps of a predetermined amount (for example, one degree) from the location on said visual field mapping image which corresponds to the fixation image display position at the time of the use of the visual field scanning apparatus.)

(Forming a part of along each concentric circle trajectory visual field mapping rectangle width average calculation means.)

Positional perception, in degrees, of blind spot, visual defect region, (visual defect region pathway from peripheral visual defect region to the blind spot,) etc becomes facilitated.

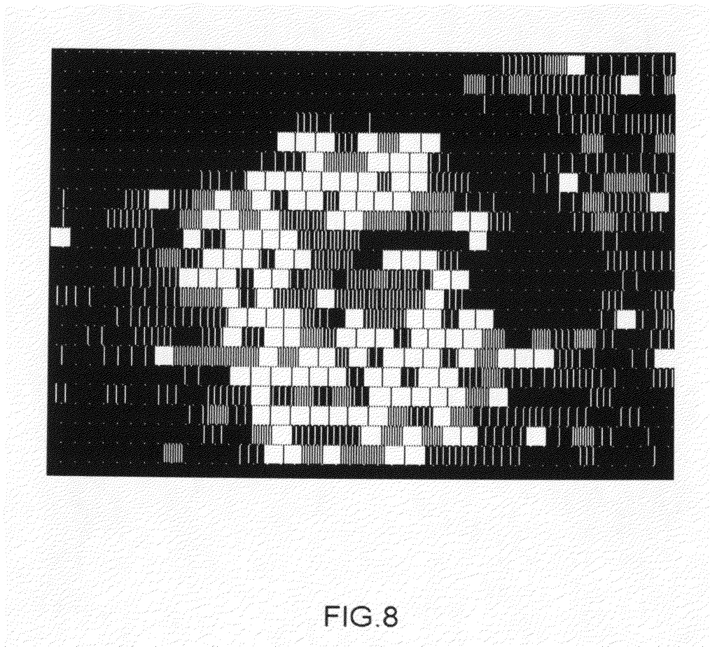

FIG. 8 is a visual field of the right eye (of FIG. 2).

Color coded according to the width of each visual field mapping rectangle.

20-21dots range with RGB(250,0,0).

22-23dots range with RGB(200,0,0).

24-25dots range with RGB(150,0,0).

26-27dots range with RGB(100,0,0).

28-29dots range with RGB(0,250,0).

30-31dots range with RGB(0,200,0).

32-33dots range with RGB(0,150,0).

34-35dots range with RGB(0,100,0).

36-37dots range with RGB(0,0,250).

38-39dots range with RGB(0,0,200).

40-41dots range with RGB(0,0,150).

42-43dots range with RGB(0,0,100).

Otherwise with RGB(0,0,0).

(e.g., 20-21dots range corresponds also to about 200-210 ms (milliseconds) range in this example.)

A tendency of (concentric) decreasing of visual sensitivity (in radial outward direction from fovea) and a tendency of parallelism of visual defect region (and visual sensitivity declining region) with retinal nerve fiber tracts are intuitively represented.

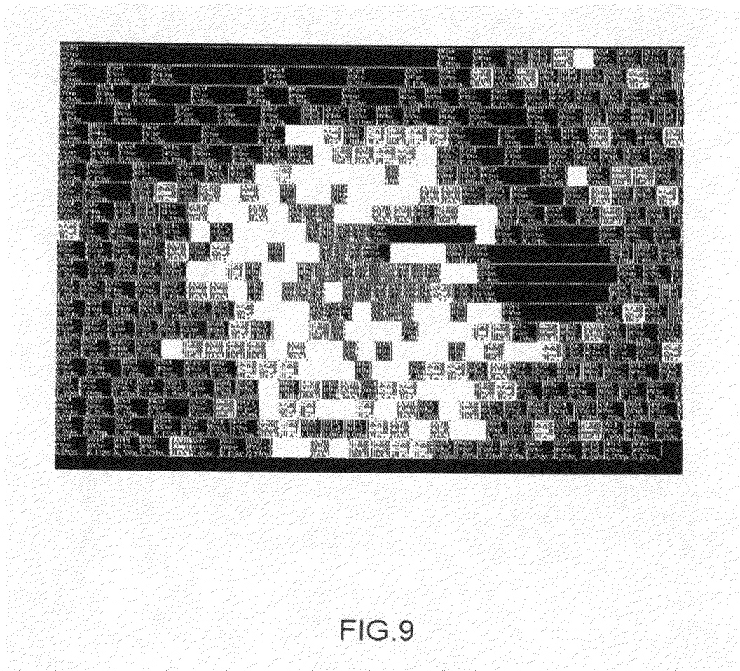

FIG. 9 is a synthesized representation from FIG. 8 and FIG. 4.

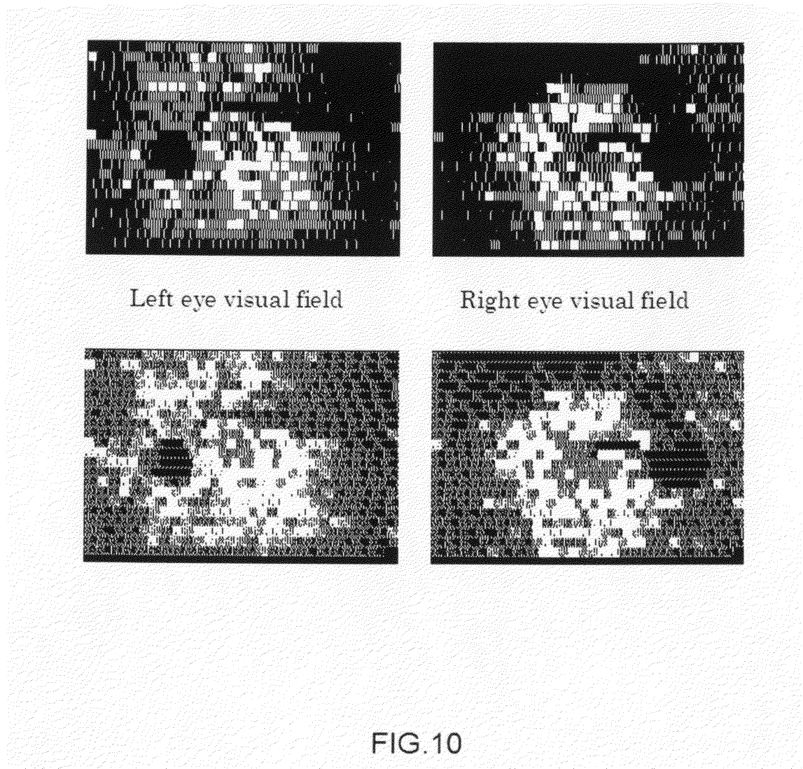

In FIG. 10, regarding FIG. 8 and FIG. 9, left eye visual field is juxtaposed to right eye visual field.

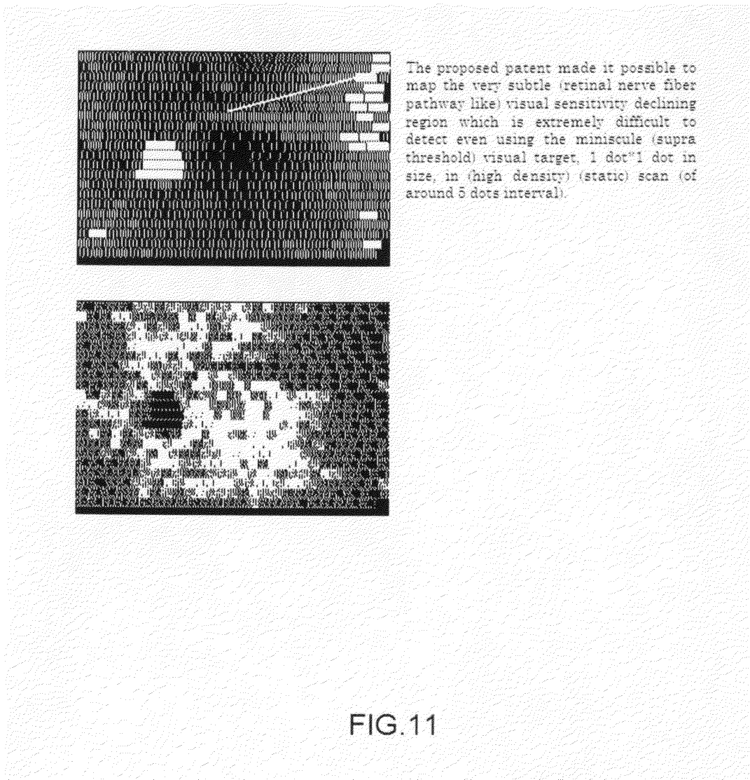

In FIG. 11, the very slight visual sensitivity declining region, which is extremely difficult to detect even using a miniscule (supra threshold) visual target, 1 dot (3 mins)*1 dot (3 mins) in size, in (high density) (static) scan (of around 5 dots (15 mins) test point interval), can become detected in the visual field mapping image of the left eye (of FIG. 11) by kinetic, successive use of a visual target by the proposed invention, although this invention (of this example) uses a relatively large visual target (with supra threshold intensity), 5 dot (15 mins)*5 dot (15 mins) in size (a visual target of supra threshold in terms of low temporal resolution).

In FIG. 11, the proposed invention made it possible to map the very subtle (retinal nerve fiber pathway like) visual sensitivity declining region which is extremely difficult to detect even using the miniscule (supra threshold) visual target, 1 dot*1 dot in size, in (high density) (static) scan (of around 5 dots interval).

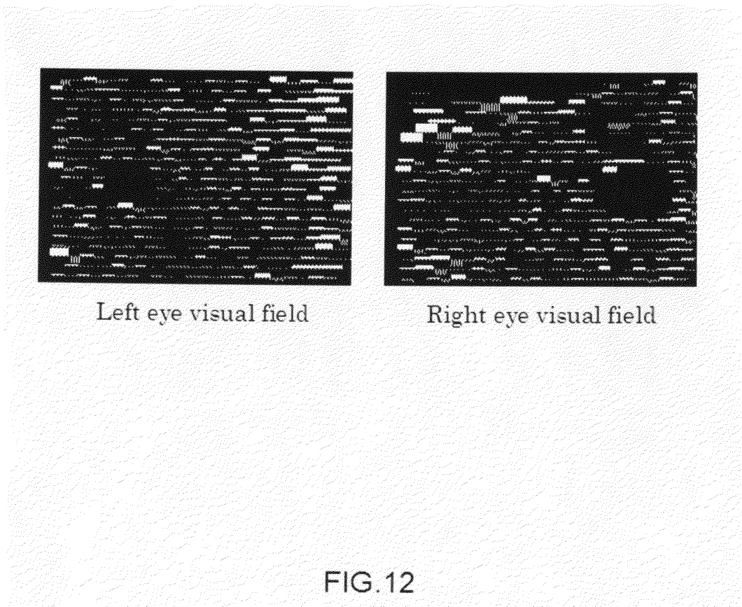

FIG. 12 is a map expressing the difference in width of (horizontally) adjacent visual field mapping rectangles, in y axis direction.

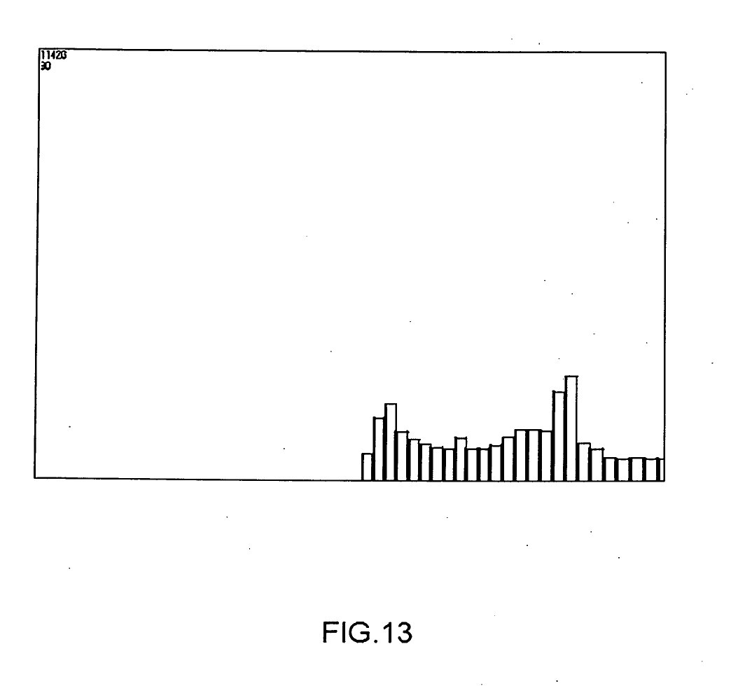

FIG. 13 is a graph representing a transition toward radial direction of the average values of visual field mapping rectangle width in terms of the first quadrant of each concentric circle trajectory (which is generated as in FIG. 7) on the right eye visual field mapping image (FIG. 2).

The x axis direction represents the radius increase from the fixation image display position, and each (concentric circle radial increment) bin (range of class) is represented in steps of one degree.

The y axis direction represents the average value of visual field mapping rectangle width along each concentric circle trajectory in terms of the first quadrant.

With respect to the visual field mapping image obtained from the use of the visual field scanning apparatus,

CPU 501 calculates visual field mapping rectangle width average along each concentric circle trajectory by generating a cluster of concentric circles by incrementing the radius to generate a concentric circle in steps of a predetermined amount from the location on said visual field mapping image which corresponds to the fixation image display position at the time of said use of the visual field scanning apparatus.

(Forming along each concentric circle trajectory visual field mapping rectangle width average calculation means.)

CPU 501 generates a graph which represents a transition from said location to the radial direction, of visual field mapping rectangle width average along said each concentric circle trajectory based on the value of visual field mapping rectangle width average along said each concentric circle trajectory which is calculated by said along each concentric circle trajectory visual field mapping rectangle width average calculation means (by CPU 501).

(Forming visual field mapping rectangle width average radial direction transition graph generating means.)

With respect to the visual field mapping image obtained from the use of the visual field scanning apparatus

CPU 501 calculates, for each quadrant of said visual filed mapping image whose origin is placed at the location on said visual field mapping image which corresponds to the fixation image display position at the time of said use of the visual field scanning apparatus, the visual field mapping rectangle width average along each concentric circle trajectory through generating a cluster of concentric circles by incrementing the radius to generate a concentric circle in steps of a predetermined amount from said location on said visual field mapping image which corresponds to the fixation image display position at the time of said use of the visual field scanning apparatus;

(Forming for each quadrant along each concentric circle trajectory visual field mapping rectangle width average calculation means.)

CPU 501 generates, for each quadrant, a graph which represents a transition from said location to the radial direction of visual field mapping rectangle width average for said each quadrant along each concentric circle trajectory based on the value of visual field mapping rectangle width average for said each quadrant along each concentric circle trajectory which is calculated by said for each quadrant along each concentric circle trajectory visual field mapping rectangle width average calculation means (by CPU 501).

(Forming for each quadrant visual field mapping rectangle width average radial direction transition graph generating means.)

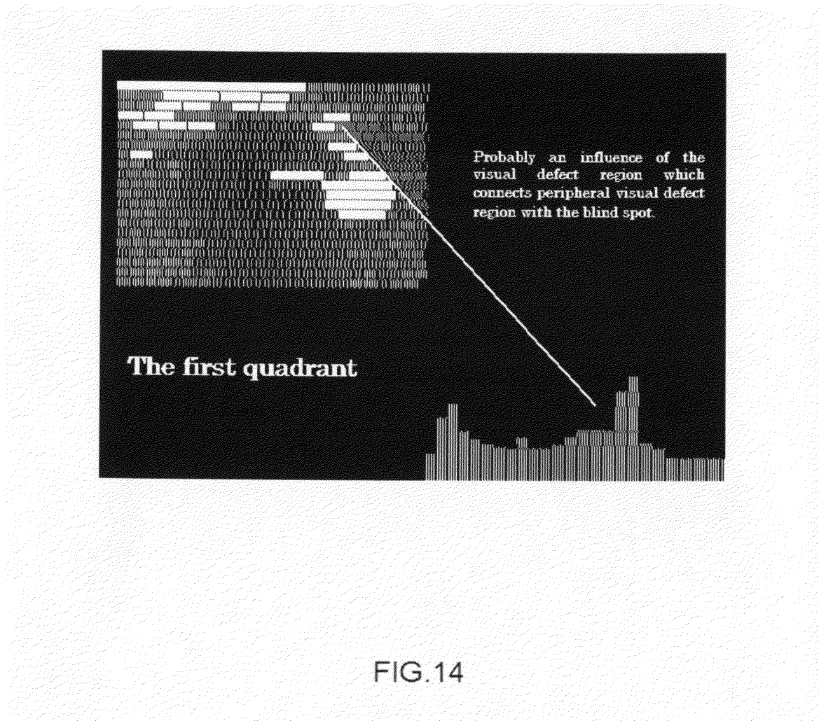

In FIG. 14, FIG. 13 (calculated for the first quadrant of the visual field) is juxtaposed to FIG. 2.

Probably an influence of the visual defect region which connects peripheral visual defect region with the blind spot is reflected.

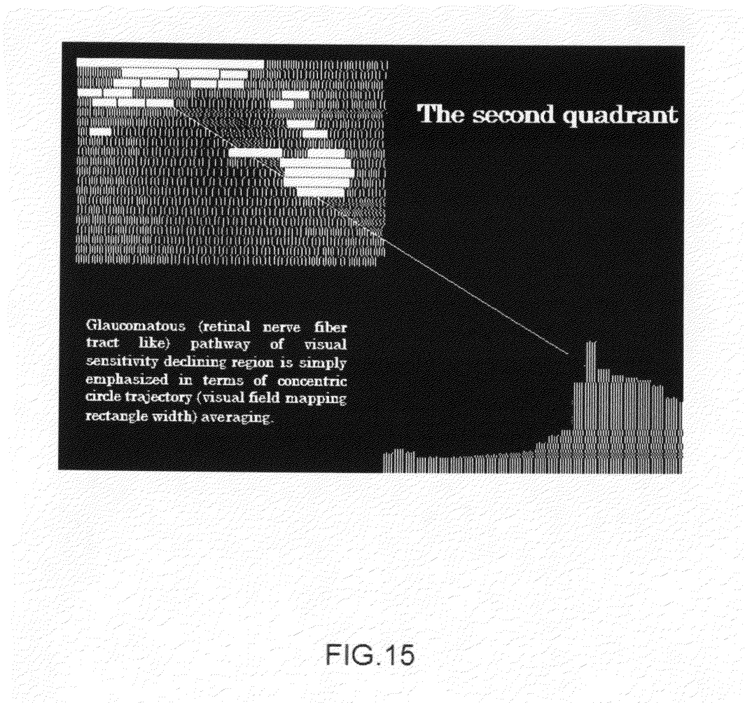

FIG. 15 is a graph representing a transition toward radial direction of the average values of visual field mapping rectangle width in terms of the second quadrant of each concentric circle trajectory on the right eye visual field mapping image (of FIG. 2) (juxtaposed to FIG. 2).

The x axis direction represents the radius increase from the fixation image display position, and each (concentric circle radial increment) bin (range of class) is represented in steps of one degree.

The x axis is in opposite direction (horizontally) to the visual field mapping image second quadrant.

The y axis direction represents the average value of visual field mapping rectangle width along each concentric circle trajectory in terms of the second quadrant.

Glaucomatous (retinal nerve fiber tract like) pathway of visual sensitivity declining region is simply emphasized in terms of concentric circle trajectory (visual field mapping rectangle width) averaging.

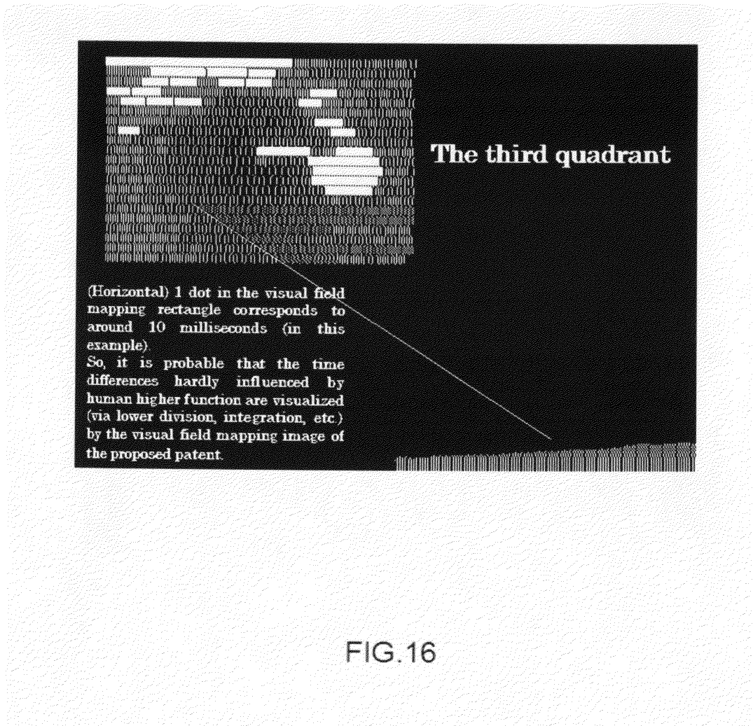

FIG. 16 is a graph representing a transition toward radial direction of the average values of visual field mapping rectangle width in terms of the third quadrant of each concentric circle trajectory on the right eye visual field mapping image (of FIG. 2) (juxtaposed to FIG. 2).

The x axis direction represents the radius increase from the fixation image display position, and each (concentric circle radial increment) bin (range of class) is represented in steps of one degree.

The x axis is in opposite direction (horizontally) to the visual field mapping image third quadrant.

The y axis direction represents the average value of visual field mapping rectangle width along each concentric circle trajectory in terms of the third quadrant.

(Horizontal) 1 dot in the visual field mapping rectangle corresponds to around 10 milliseconds (in this example).

So, it is probable that the time differences hardly influenced by human higher function are visualized (via lower division, integration, etc.) by the visual field mapping image of the proposed invention.

Among the right eye visual field, the third quadrant seems to be relatively normal in visual sensitivity (and the radial transition), but its periphery seems to have some tendency of slight (glaucomatous) visual sensitivity declining.

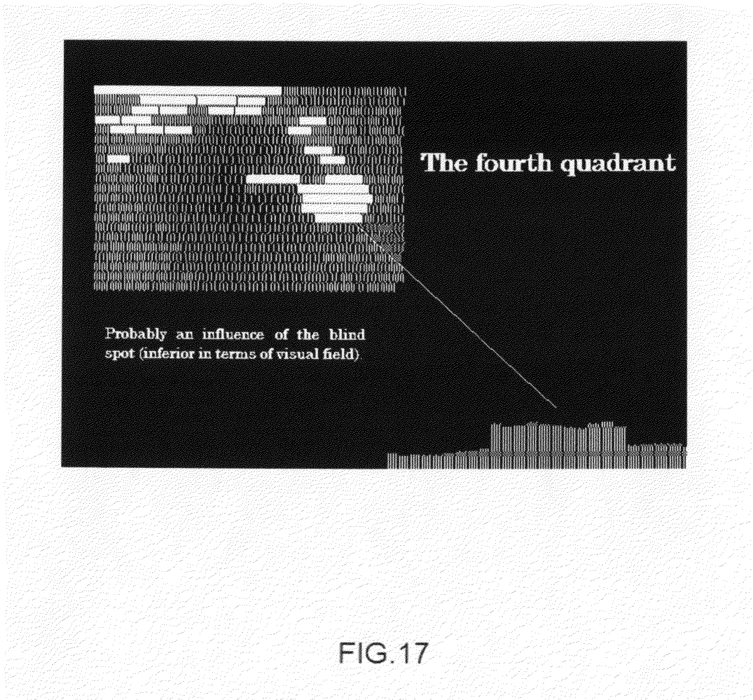

FIG. 17 is a graph representing a transition toward radial direction of the average values of visual field mapping rectangle width in terms of the fourth quadrant of each concentric circle trajectory on the right eye visual field mapping image (of FIG. 2) (juxtaposed to FIG. 2).

The x axis direction represents the radius increase from the fixation image display position, and each (concentric circle radial increment) bin (range of class) is represented in steps of one degree.

The y axis direction represents the average value of visual field mapping rectangle width along each concentric circle trajectory in terms of the fourth quadrant.

Probably an influence of the blind spot (inferior in terms of visual field) is reflected.

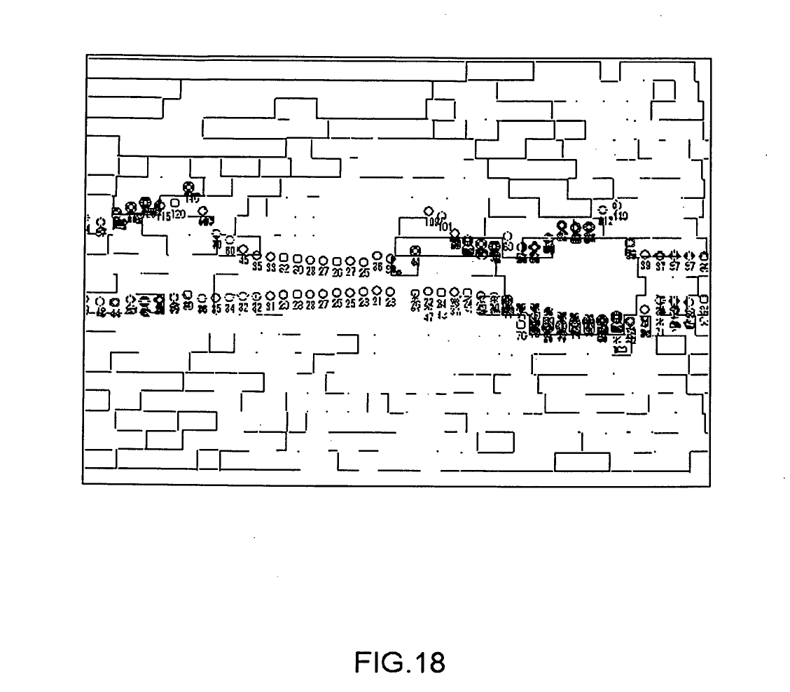

In FIG. 18, each quadrant visual field mapping rectangle width (concentric) average radial direction transition graph calculated using a program different (in sampling, etc.) from FIGS. 14-17 is shown simultaneously for all the quadrants (overlaid on the visual field mapping image).

In this figure, the x axis direction in visual field mapping rectangle width (concentric) average radial direction transition graph coincides with the x axis direction in the visual field mapping image, for all the quadrants.

The visual field mapping rectangle width (concentric) average radial direction transition graph represented by asterisk in the fourth quadrant of FIG. 18 is the visual field mapping rectangle width (concentric) average radial direction transition graph calculated for all the quadrants.

With respect to the visual field mapping image obtained from the use of the visual field scanning apparatus,

CPU 501 calculates visual field mapping rectangle width average along each concentric circle trajectory by generating a cluster of concentric circles by incrementing the radius to generate a concentric circle in steps of a predetermined amount from the location on said visual field mapping image which corresponds to the fixation image display position at the time of said use of the visual field scanning apparatus.

(Forming along each concentric circle trajectory visual field mapping rectangle width average calculation means.)

CPU 501 generates a graph which represents a transition from said location to the radial direction, of visual field mapping rectangle width average along said each concentric circle trajectory based on the value of visual field mapping rectangle width average along said each concentric circle trajectory which is calculated by said along each concentric circle trajectory visual field mapping rectangle width average calculation means (by CPU 501).

(Forming visual field mapping rectangle width average radial direction transition graph generating means.)

With respect to the visual field mapping image obtained from the use of the visual field scanning apparatus,

CPU 501 calculates, for each quadrant of said visual filed mapping image whose origin is placed at the location on said visual field mapping image which corresponds to the fixation image display position at the time of said use of the visual field scanning apparatus, the visual field mapping rectangle width average along each concentric circle trajectory through generating a cluster of concentric circles by incrementing the radius to generate a concentric circle in steps of a predetermined amount from said location on said visual field mapping image which corresponds to the fixation image display position at the time of said use of the visual field scanning apparatus;

(Forming for each quadrant along each concentric circle trajectory visual field mapping rectangle width average calculation means.)

CPU 501 generates, for each quadrant, a graph which represents a transition from said location to the radial direction of visual field mapping rectangle width average for said each quadrant along each concentric circle trajectory based on the value of visual field mapping rectangle width average for said each quadrant along each concentric circle trajectory which is calculated by said for each quadrant along each concentric circle trajectory visual field mapping rectangle width average calculation means (by CPU 501).

(Forming for each quadrant visual field mapping rectangle width average radial direction transition graph generating means.)

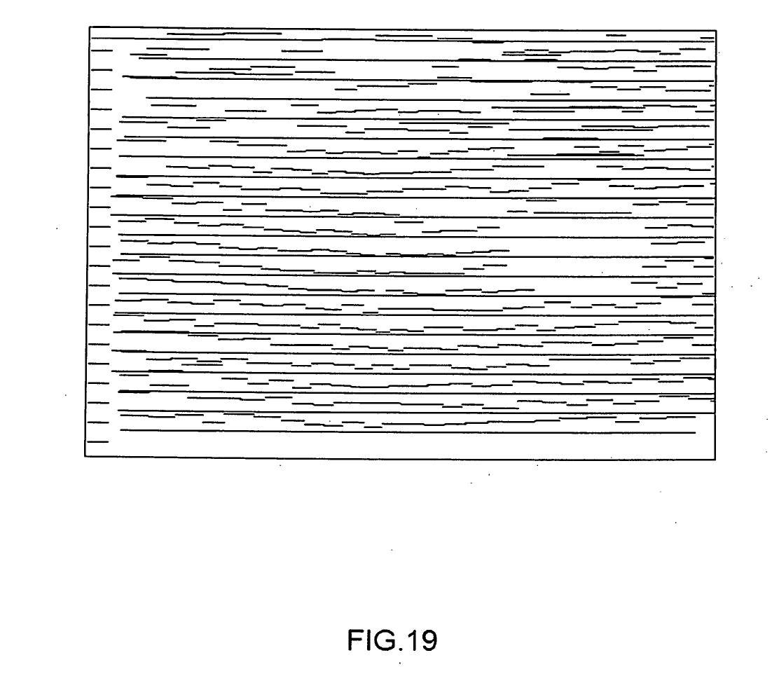

FIG. 19 is a representation expressing in y axis direction the width of each visual field mapping rectangle in the right eye visual field mapping image (of FIG. 2).

This is thought to be (two dimensional (defined by the direction of a visual target scan line (rightward direction) and the direction normal to the visual field)) cross sections, at each (horizontal) visual target scan line, of the visual sensitivity three dimension, of the proposed invention, that expresses visual sensitivity variable (visual field mapping rectangle width) at each rectangle location, in the direction vertical to the visual field.

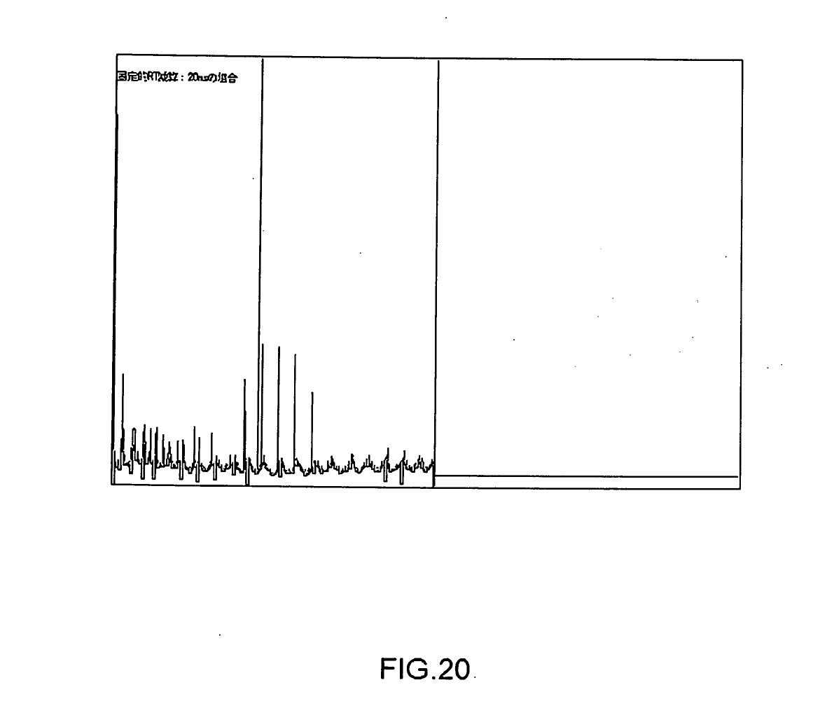

FIG. 20 is a graph regarding the right eye visual field mapping image (of FIG. 2);

In the x axis direction, 1 visual field mapping rectangle is represented by 1 dot.

(In the x axis direction, visual field mapping rectangle width is not considered.)

Visual field mapping rectangles are sequentially (rightward within a (horizontal) visual target scan line and downward in units of visual target scan line) represented as (x axis directional) dots (in the x axis direction).

The y axis direction represents the visual field mapping rectangle width (in units of dot) (corresponding to each visual field mapping rectangle represented as a (x axis directional) dot in the x axis direction).

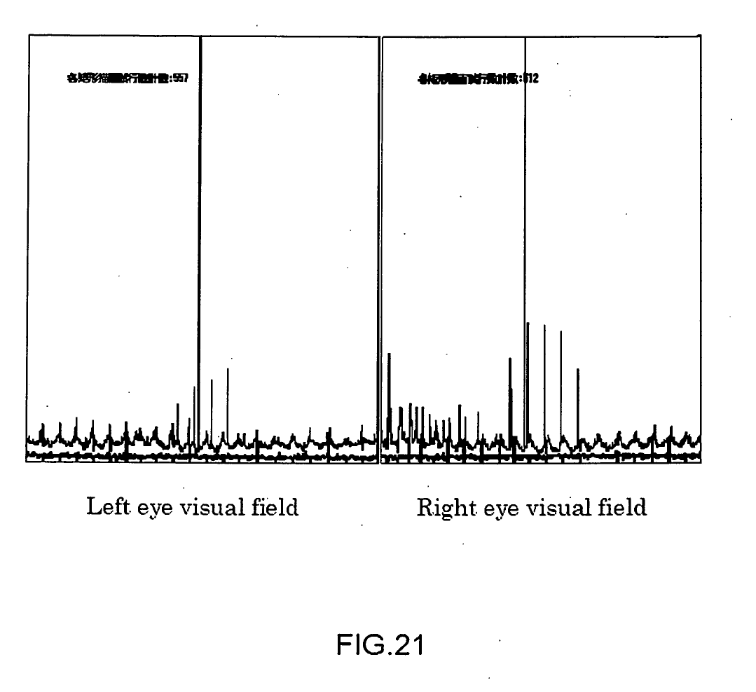

In FIG. 21, the corresponding graph for the left eye visual field is juxtaposed to FIG. 20 for the right eye visual field.

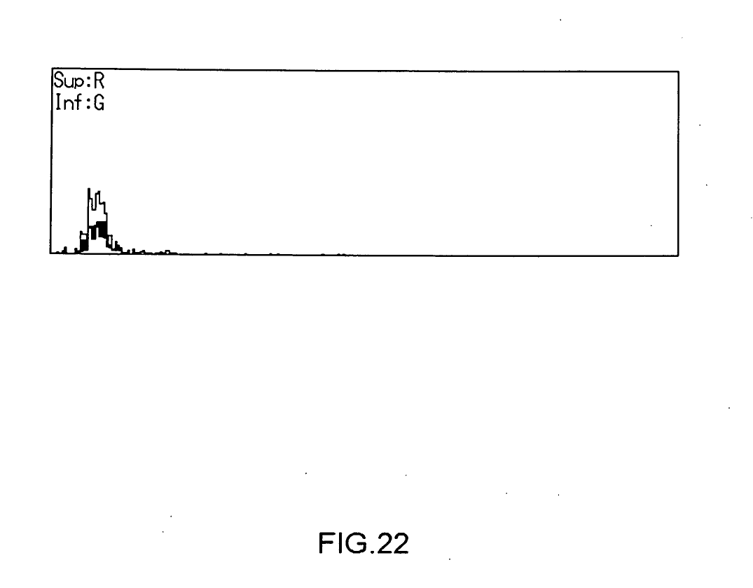

FIG. 22 is a (class) frequency distribution graph regarding the right eye visual field mapping image (of FIG. 2);

The x axis direction represents the (visual field mapping rectangle width) class (bin) increase direction (i.e., visual sensitivity decline direction).

Each class range (bin) is 2 dots.

The y axis direction represents the frequency counted for each class (bin).

Red: the frequency (component) with respect to superior visual field.

Green: the frequency (component) with respect to inferior visual field.

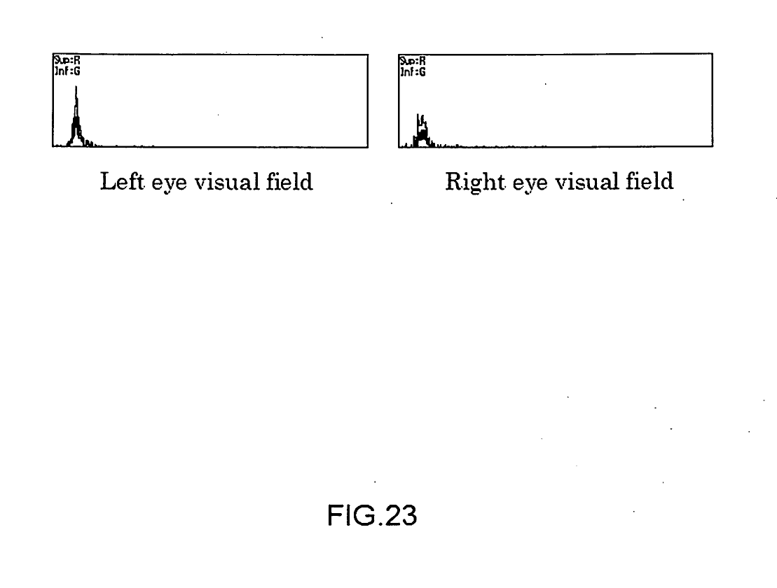

In FIG. 23, the shape of mode vicinity is more eroded (degraded) in the right eye visual field compared to the left probably due to severer (glaucomatous) visual sensitivity decline of the right eye visual field compared to the left.

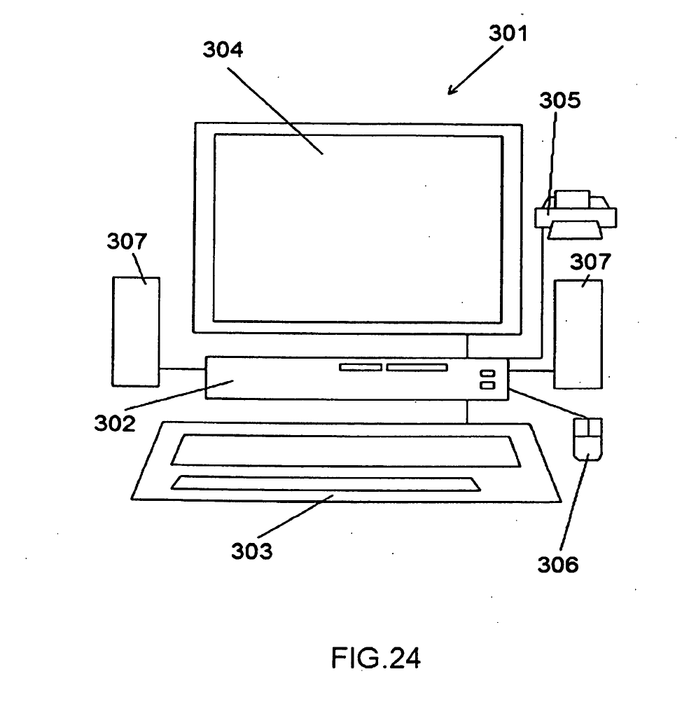

FIG. 24 shows a computer system 301 diagrammatically.

The present invention is realized by the computer system 301 carrying out a program for realizing the present invention.

As shown in FIG. 24, the computer system 301 realizing an embodiment of the present invention includes a main unit 302 that is equipped with a CPU (Central Processing Unit) 501, etc., which will be mentioned later, a keyboard 303, (if necessary, a mouse 306), a display 304, (and a printer 305) (and if necessary, a speaker 307 too).

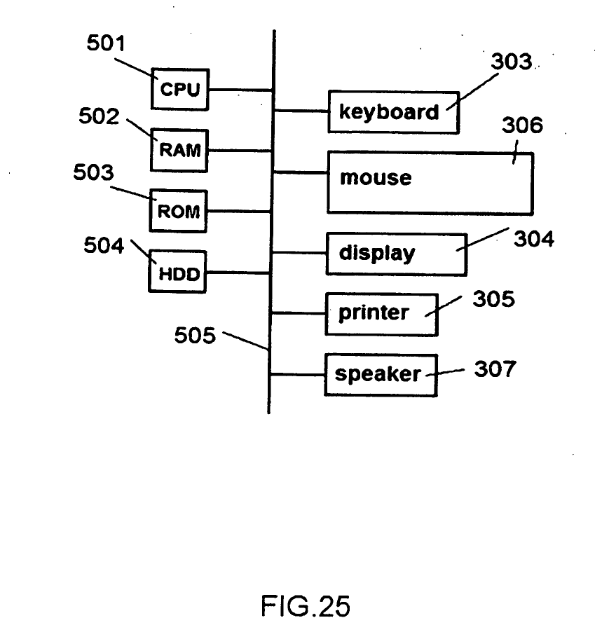

Next, an embodiment of the hardware configuration of the CPU 501 in the present invention is described referring to FIG. 25.

The CPU 501 in the present invention is configured specifically including: a microprocessor such as the CPU 501, a RAM (Random Access Memory) 502, a ROM (Read Only Memory) 503, a HDD (Hard Disc Drive) 504, a keyboard 303, a mouse 306, a display 304, a printer 305, a speaker 307, and a communications interface.

These parts are connected via a bus 505.

The CPU 501 carries out operations characteristic of an embodiment of the present invention, by loading onto the RAM 502 a program, which is stored in the HDD (Hard Disc Drive) 504, for realizing the present invention.

The CPU 501 carries out controls, and kinds of arithmetic processing, of the present invention, according to a program for realizing the present invention.

The CPU 501 controls display processing of the display 304 (an example of the output device).

The CPU 501 controls the present invention according to input by the keyboard 303 (an example of the input device).

The CPU 501 can control the printer 305 and the like so as to output the visual field mapping image, etc. that are generated based on the data obtained from the present invention.

The keyboard 303 (and if necessary, the mouse 306) and the display 304 are used as user interfaces in the present invention.

The keyboard 303 is used, for example, as a device for input (the input device).

(If necessary, the mouse 306 is used as a device for performing various kinds of operations of input to the display screen of the display 304.)

The display 304 is a display device (the output device), for example, of a LCD (Liquid Crystal Display), a CRT (Cathode Ray Tube), an OLED (Organic Light-Emitting Diode), or the like, which displays a visual field mapping image generated by the present invention.

And when the CPU 501 is connected to communications network such as the Internet and a LAN (Local Area Network), the communications interface can be equipped with a network adapter such as a LAN card or communications equipment such as a modem, in order to establish data communication among the network. In such a case, by installing on the network a server storing a program for realizing the present invention, and configuring the CPU 501 as a client terminal of the server, the operation of the present invention can be carried out by the apparatus.

A program for realizing the present invention can be stored on any computer-readable non-transitory media (storage media).

Examples of such non-transitory media (storage media) are an optical disk, a magneto-optic disk (CD-ROM, DVD-RAM, DVD-ROM, MO, etc.), a magnetic-storage device (hard disk, Floppy Disk™, ZIP, etc.), a semiconductor memory, etc.

Paracentral scotoma 201 is a paracentral scotoma.

Connecting portion between scotoma and the blind spot 202 is a connecting portion between scotoma and the blind spot, such as the visual defect region connecting the peripheral visual defect to the blind spot.

Blind spot 203 is the (physiological) blind spot (corresponding to the optic disc).

Visual function declining region 204 is a visual function declining region.

Visual function slightly declining region 205 is a visual function slightly declining region.

Fixation image display position 206 is a fixation image display position.

Fovea 207 is (the location corresponding to) the fovea.

Visual function very slightly declining region 220 is a visual function very slightly declining region.

The visual field scanning apparatus (in this invention) may refer to any of the following;

U.S. Pat. No. 7,993,002

U.S. Pat. No. 8,083,352

U.S. Pat. No. 8,020,996

WO2010/026890

WO2010/024010

WO2010/032592

A visual field visual function mapping apparatus may be, with respect to the visual field mapping image obtained from the use of the visual field scanning apparatus, characterized by further comprising:

along each (of groups of) retinal nerve fiber(s) (bundle(s)) trajectory visual field mapping rectangle width average calculation means to calculate visual field mapping rectangle width average along each (of groups of) retinal nerve fiber(s) (bundle(s)) trajectory by referring to each (of groups of) retinal nerve fiber(s) (bundle(s) (which may become directly observable with AOSLO (adaptive optics scanning laser ophthalmoscopy) or AOOCT (adaptive optics optical coherence tomography), or which may be approximately found in some (medical statistics) normative data);

and visual field mapping rectangle width average spatial transition graph generating means to generate a graph which represents a spatial transition of visual field mapping rectangle width average along said each (of groups of) retinal nerve fiber(s) (bundle(s)) trajectory based on the value of visual field mapping rectangle width average along said each (of groups of) retinal nerve fiber(s) (bundle(s)) trajectory which is calculated by said along each (of groups of) retinal nerve fiber(s) (bundle(s)) trajectory visual field mapping rectangle width average calculation means.

What is claimed is:

1. A visual field visual function mapping apparatus is, with respect to the visual field mapping image obtained from the use of the visual field scanning apparatus, characterized by further comprising:
along each concentric circle trajectory visual field mapping rectangle width average calculation means to calculate visual field mapping rectangle width average along each concentric circle trajectory by generating a cluster of concentric circles by incrementing the radius to generate a concentric circle in steps of a predetermined amount from the location on said visual field mapping image which corresponds to the fixation image display position at the time of said use of the visual field scanning apparatus; and
visual field mapping rectangle width average radial direction transition graph generating means to generate a graph which represents a transition from said location to the radial direction of visual field mapping rectangle width average along said each concentric circle trajectory based on the value of visual field mapping rectangle width average along said each concentric circle trajectory which is calculated by said along each concentric circle trajectory visual field mapping rectangle width average calculation means.

2. A visual field visual function mapping apparatus is, with respect to the visual field mapping image obtained from the use of the visual field scanning apparatus, characterized by further comprising:
for each quadrant along each concentric circle trajectory visual field mapping rectangle width average calculation means to calculate, for each quadrant of said visual filed mapping image whose origin is placed at the location on said visual field mapping image which corresponds to the fixation image display position at the time of said use of the visual field scanning apparatus, the visual field mapping rectangle width average along each concentric circle trajectory through generating a cluster of concentric circles by incrementing the radius to generate a concentric circle in steps of a predetermined amount from said location on said visual field mapping image which corresponds to the fixation image display position at the time of said use of the visual field scanning apparatus; and
for each quadrant visual field mapping rectangle width average radial direction transition graph generating means to generate, for each quadrant, a graph which represents a transition from said location to the radial direction of visual field mapping rectangle width average for said each quadrant along each concentric circle trajectory based on the value of visual field mapping rectangle width average for said each quadrant along each concentric circle trajectory which is calculated by said for each quadrant along each concentric circle trajectory visual field mapping rectangle width average calculation means.
